(19) United States
(12) Patent Application Publication
(10) Pub. No.: US 2015/0366836 A1
(43) Pub. Date: Dec. 24, 2015

(54) METHOD AND PHARMACEUTICAL COMPOSITION FOR TREATING COLORECTAL CANCER

(71) Applicant: National Dong Hua University, Hualien (TW)

(72) Inventors: Ching-Feng Weng, Hualien (TW); Yi-Chen Chia, Hualien (TW); Jia-Huei Zheng, Hualien (TW)

(21) Appl. No.: 14/313,852

(22) Filed: Jun. 24, 2014

(51) Int. Cl. A61K 31/341; A61K 49/00; A61K 31/513

(52) U.S. Cl. CPC A61K 31/341; A61K 31/513; A61K 49/0008

(57) ABSTRACT

The present invention is related to a method and pharmaceutical composition for treating colorectal cancer. The pharmaceutical composition comprises an effective amount of 16-hydroxy-cleroda-3,13-dine-15,16-olide as active ingredient, and a pharmaceutically acceptable carrier. The present method and pharmaceutical composition provides good efficacy in treating colorectal cancer. The present invention also establishes an animal model, which provides a better drug screening platform for the research.

### BACKGROUND

1. Technical Field

The present disclosure relates to a method and a pharmaceutical composition for treating colorectal cancer. In addition, the present invention relates to the establishment of animal model.

2. Description of Related Art

Colorectal cancer (including colon cancer and rectum cancer) is malignant tumor that comes from polypus in the colon. The mortality rate is the third place of all cancers in Taiwan when the patient is diagnosed with colorectal cancer in the later period or metastasis with low survival rate. Colorectal cancer proliferating to colon or rectal is characterized by cell accumulation near lymph nodes, and those cells accumulated near lymph nodes may invade and migrate through the lymphatic system to other organs or tissues such as liver.

As appreciated the difficulty in treating colorectal cancer, it is the first priority goal of the field to find novel and effective drugs. Furthermore, there is always a distinct gap between laboratory experiments and clinical use; therefore, it is also an important task to imitate the laboratory experiments as similar as possible with a realistic pathophysiological condition. In this consideration, animal model is indeed taken as a significant step before the candidate drugs actually enter clinical trials. Of course, the animal model itself has still certain level of difference from human body. However, the appropriate animal model can provide a suitable glance to mimic for the exploration of human body disease.

In light of the foregoing, there is always a need for novel and useful drug for treating colorectal cancer. In addition, it will be helpful for the drug screening if the conventional animal model can be modified to be more similar to human body.

### SUMMARY

One of the objects of the present invention is to provide a novel and useful drug for treating colorectal cancer by validating the medical use of 16-hydroxy-cleroda-3,13-dine-15,16-olide (HCD) in this regard.

Another object of the present invention is to provide a method for treating colorectal cancer, preferably the method can align the use of 16-hydroxy-cleroda-3,13-dine-15,16-olide with other conventional anti-cancer drugs to reduce the required dosage of said conventional anti-cancer drugs and obtain better efficacy.

More an object of the present invention is to establish an animal model having pathophysiological condition more similar to human.

In order to achieve the aforesaid objects, the present invention provides a pharmaceutical composition for colorectal cancer treatment, comprising: an effective amount of 16-hydroxy-cleroda-3,13-dine-15,16-olide; and a pharmaceutically acceptable carrier.

Preferably, said effective amount is 0.6 to 6.5 mg/kg body weight.

Preferably, said composition comprises 0.5 to 10 μM of said 16-hydroxy-cleroda-3,13-dine-15,16-olide.

Preferably, said pharmaceutically acceptable carrier is water, phosphate buffered saline, alcohol, glycerol, chitosan, alginate, chondroitin, Vitamin E, mineral oil, dimethyl sulfoxide (DMSO), or a combination thereof.

Preferably, an administration route of said composition is via oral administration, intravenous injection, intrathecal injection, intraperitoneal injection, or a combination thereof.

The present invention also provides a method for treating a colorectal cancer, comprising: administrating an object in need an effective amount of 16-hydroxy-cleroda-3,13-dine-15,16-olide.

Preferably, said effective amount is 0.6 to 6.5 mg/kg body weight.

Preferably, said administrating is via oral administration, intravenous injection, intrathecal injection, intraperitoneal injection, or a combination thereof.

Preferably, said 16-hydroxy-cleroda-3,13-dine-15,16-olide is administrated with a pharmaceutically acceptable carrier

Preferably, said pharmaceutically acceptable carrier is water, phosphate buffered saline, alcohol, glycerol, chitosan, alginate, chondroitin, Vitamin E, mineral oil, dimethyl sulfoxide (DMSO), or a combination thereof.

Preferably, said method further comprises a step after administrating said 16-hydroxy-cleroda-3,13-dine-15,16-olide: administrating said object with 5-Fluorouracil.

The present invention more provides a method for establishing an animal model bearing an enteritis: (a) providing an animal; (b) administrating said animal with azoxymethane; and (c) administrating said animal with a dextran sodium sulfate solution.

Preferably, said administrating in step (b) is an intraperitoneal injection.

Preferably, a dosage of said azoxymethane in step (b) is 8 to 12 mg/kg body weight.

Preferably, said administrating in step (c) is oral administration via drinking water.

Preferably, said dextran sodium sulfate solution in step (c) has a concentration of 1 to 3 wt %.

Preferably, said step (c) is repeated at least once, and said method further comprises a resting period between repeats of said step (c); wherein said resting period is referred to as a period that said animal is not administrated with said azoxymethane and said dextran sodium sulfate solution.

Preferably, said enteritis is inflammatory bowel disease.

Preferably, said animal is rabbit, pig or rodent.

To sum up, the present invention validates the medical use of HCD in treating colorectal cancer and its ability to reduce required dosage of conventional anti-cancer drugs. Moreover, the present invention also establishes an animal model bearing enteritis and being patho-physiologically more similar to human than conventional animal models so that the drug screening using said animal model can be more effective and reliable in subsequent clinical trials.

### BRIEF DESCRIPTION OF THE DRAWINGS

FIG. 1 shows the results of MTT assay. The MTT assay was performed for examining the effects of 5-Fluorouracil (5-FU) on the cell viability of Caco2 cells and HT-29 cells at 24, 36, or 48 hr treatment. All the experiments were done in three independent tests and the data was presented as mean±SD in triplicate determinations. Compared at *p<0.05, **p<0.01, ***p<0.001 levels with untreated control.

FIG. 2 shows the results of MTT assay. The MTT assay was performed for examining the effects of PG on the cell viability of Caco2 cells and HT-29 cells at 24, 36, or 48 hr treatment. All the experiments were done in three independent tests and the data was presented as mean±SD in triplicate determinations. Compared at *p<0.05, **p<0.01, ***p<0.001 levels with untreated control.

FIG. 3 shows the results of MTT assay. The MTT assay was performed for examining the effects of HCD on the cell viability of Caco2 cells and HT-29 cells at 24, 36, or 48 hr treatment. All the experiments were done in three independent tests and the data was presented as mean±SD in triplicate determinations. Compared at *p<0.05, **p<0.01, ***p<0.001 levels with untreated control.

FIG. 4 shows the results of MTT assay. The MTT assay was performed for examining the effects of combining pre-treatment of PG (0.5, 2, 5 μM) and follow-up treatment of 5-FU (2 μM) on cell viability of Caco2 cells and HT-29 cells. The time of pre-treatment was 6, 12, 24, 36, or 48 hr; and the time of 5-FU treatment was 24 hr. All the experiments were done in three independent tests and the data was presented as mean±SD in triplicate determinations. Compared at *p<0.05, **p<0.01, ***p<0.001 levels with untreated control. The “c” indicated the control group without pre-treatment. The viability of control group was taken as 100%.

FIG. 5 shows the results of MTT assay. The MTT assay was performed for examining the effects of combining pre-treatment of HCD (0.5, 2, 5 μM) and follow-up treatment of 5-FU (2 μM) on cell viability of Caco2 cells and HT-29 cells. The time of pre-treatment was 6, 12, 24, 36, or 48 hr; and the time of 5-FU treatment was 24 hr. All the experiments were done in three independent tests and the data was presented as mean±SD in triplicate determinations. Compared at *p<0.05, **p<0.01, ***p<0.001 levels with untreated control. The “c” indicated the control group without pre-treatment. The viability of control group was taken as 100%.

FIG. 6 shows the body weight changing curves of normal mouse, and mouse treated with azoxymethane and dextran sodium sulfate solution during the induction procedure.

FIG. 7 shows the morphology of the colorectal portion of control mouse (a) and experimental mouse (b). The arrows indicate the polypus and enlargement lymph nodes.

FIG. 8 shows the H&E staining (40×) of the intestine of normal mice (a) and the experimental mice (b) by indicating the intestine crypts, mucosa layer, muscle layer mucosa, tunica sub-mucosa and muscle layer thereof.

FIG. 9 shows the H&E staining (40×) of the intestine of normal mice (a) and the experimental mice (b) by indicating the lymph node thereof.

FIG. 10 shows the body weight changing curves of normal mouse (control), mouse bearing IBD (AOM/DSS induced), mouse bearing IBD and treated with 5-FU, and mouse bearing IBD and treated with HCD.

FIG. 11 shows the H&E staining of the intestine indicating the intestine crypts (arrows). (a) mouse treated with 15 mg/kg body weight 5-FU (40×); (b) mouse treated with 0.64 mg/kg body weight HCD (40×); (c) mouse treated with 1.6 mg/kg body weight HCD (40×); and (d) mouse treated with 6.4 mg/kg body weight HCD (30×).

FIG. 12 shows the H&E staining of the intestine indicating the lymph node (arrows). (a) mouse treated with 15 mg/kg body weight 5-FU (40×); (b) mouse treated with 0.64 mg/kg body weight HCD (40×); (c) mouse treated with 1.6 mg/kg body weight HCD (40×); and (d) mouse treated with 6.4 mg/kg body weight HCD (30×).

### DETAILED DESCRIPTION

16-hydroxy-cleroda-3,13-dine-15,16-olide (HCD) isolated from Polyalthia longifolia possess some medicinal values; however, there is no evidence showing its value in treating colorectal cancer before the present invention. The term “colorectal cancer” herein is referred to as colon cancer, rectum cancer, or a combination thereof.

The term “treatment or treating” herein is referred to control or reduce the size of the tumor, prevent or limit the metastasis of the cancer cells, or a combination thereof. The term “effective amount” herein is referred to as an amount of the active ingredient that is sufficient to perform the aforesaid efficacies of treatment.

Said effective amount can be obtained from clinical trial, animal model, or in vitro cell culture data. It is known in the field that the effective amount obtained from animal model or in vitro cell culture data can be calculated into the effective amount suitable for human use. For instance, as reported by Reagan-Shaw et al., 2008, “μg/ml” (effective amount based on in vitro cell culture experiments)=“mg/kg body weight/day” (effective amount for mouse). Furthermore, the effective amount for mouse can be further modified based on the fact that the metabolism rate of mice is 6 times fast compared to human.

Said pharmaceutically acceptable carrier in the present invention includes but not limited to water, phosphate buffered saline, alcohol, glycerol, chitosan, alginate, chondroitin, Vitamin E, mineral oil, dimethyl sulfoxide (DMSO), or a combination thereof. Generally, the pharmaceutically acceptable carrier can be chosen based on the desired administration route, components of the drug, treatment strategies, or purposes to be met.

The first aspect of the present invention is to provide a pharmaceutical composition for colorectal cancer treatment. Said pharmaceutical composition comprises 16-hydroxy-cleroda-3,13-dine-15,16-olide (HCD) as the active ingredient. The effective amount of said HCD is 0.6 to 6.5 mg/kg body weight. The pharmaceutical composition can be administrated via oral administration, intravenous injection, intrathecal injection, intraperitoneal injection, or a combination thereof.

The second aspect of the present invention is to provide a method for treating a colorectal cancer, comprising: administrating an object in need an effective amount of 16-hydroxy-cleroda-3,13-dine-15,16-olide. The effective amount of said HCD is 0.6 to 6.5 mg/kg body weight. The administrating can be via oral administration, intravenous injection, intrathecal injection, intraperitoneal injection, or a combination thereof.

In a preferable embodiment of the present invention, a treating strategy is provided. Said treating strategy is to reduce the required dosage of a conventional anti-cancer drug. Said treating strategy comprises a pre-treatment and a subsequent treatment. Said pre-treatment is administrating an object in need an effective amount of 16-hydroxy-cleroda-3,13-dine-15,16-olide. Said subsequent treatment is administrating said object with a conventional anti-cancer drug. After said pre-treatment, the data of the present invention showed that the efficacy of said anti-cancer drug can be improved and the dosage required for the efficacy can be reduced.

Taking 5-Fluorouracil (5-FU, which is a known drug for chemotherapy for colorectal cancer) as an example, the present invention showed (in the following data) pre-treating with HCD can enhance the efficacy of the subsequent 5-FU treatment. The data further showed that the pre-treatment of Prodigiosin (PG, as a positive) can also provide similar effects in enhancing the efficacy of the subsequent 5-FU treatment. Accordingly, the present invention indicates the potential of a co-treating strategy having a pre-treatment of HCD or PG and a subsequent treatment of an anti-cancer drug.

The third aspect of the present invention is to establish an animal model bearing enteritis. In a preferable embodiment of the present invention, said animal is not immunodeficient, which means said animal is normal in immunological competence. In this way, variances interfering with the experiments can be reduced and the drug screening data from said animal model can be more likely to be the things happened in human body. In an alternative embodiment, said animal can be rabbit, pig or rodent.

In a preferable embodiment, after said animal is ready, the present method for establishing said animal model can be separated into three periods of induction:

The first period is to administrate said animal with azoxymethane (AOM). Said azoxymethane can be administrated by intraperitoneal injection. Preferably, a dosage of said azoxymethane is 8 to 12 mg/kg body weight.

The second period is to administrate said animal with a dextran sodium sulfate solution (DSS solution). Preferably, said dextran sodium sulfate solution is taken as the daily drinking water of said animal, which means said dextran sodium sulfate solution is administrated by oral administration. Preferably, said dextran sodium sulfate solution in step (c) has a concentration of 1 to 3 wt %.

The third period is a resting period. Said resting period is referred to as a period that said animal is not administrated with said azoxymethane and said dextran sodium sulfate solution.

In a preferable embodiment of the present invention, the second period is repeated at least once and said resting period is conducted between repeats of said second period. Said “conducted between repeats” is referred to that the resting period can be conducted between every repeat of said second period or between preceding repeats and subsequent repeats.

For instance, said second period of administrating said animal with a dextran sodium sulfate solution is conducted once a day for 7 successive days, which is recognized as said second period is repeated 7 times. Then, said third period (resting period) is conducted to “rest” said animal for 7 successive days. After that, another said second period was conducted for additional 7 successive days.

### Example 1

### Experimental Design

### Reagent

The 16-hydroxy-cleroda-3,13-dine-15,16-olide (HCD) used in the present study was obtained from Professor Yi-Chen Chia (Department of Food Science & Technology, Tajen University, Taiwan). The Prodigiosin (PG) isolated from Serratia marcescens was obtained from Professor Jui-Hsin Su (Institute of Marine Biotechnology, National Drug Hwa University, Taiwan). The known drug for chemotherapy, 5-Fluorouracil (5-FU) was purchased from Sigma. Said HCD, PG, and 5-FU of various concentrations were dissolved in phosphate buffered saline (PBS) as indicated in the following paragraphs and were sterilized before use.

### Cells

Two cell lines were used in this study, Caco2 cells and HT-29 cells. Both of them are colorectal cancer cells. Caco2 cells and HT-29 cells were maintained in DMEM (or RPMI) supplemented with 20% or 10% fetal bovine serum (FBS; GIBCO), pH 7.4 at 37° C. with continuous circulation of 5% CO₂ incubator. The medium was changed every 2 days and the cells were trypsinized using trypsin/EDTA when reaching 80%-90% confluence.

### Treatments

Caco2 cells and HT-29 cells were treated by the following treatment (Table 1) for 24, 36 or 48 hr for examining the effects of HCD, PG, and Dox on their viability.

FU1 is referred as FU of 1 μM; FU2 is referred as FU of 2 μM; FU10 is referred as FU of 10 μM; FU50 is referred as FU of 50 μM; FU100 is referred as FU of 100 μM.

HCD0.5 is referred as HCD of 0.5 μM; HCD1 is referred as HCD of 1 μM; HCD2 is referred as HCD of 2 μM; HCD5 is referred as HCD of 5 μM; HCD10 is referred as HCD of 10 μM.

PG0.5 is referred as PG of 0.5 μM; PG1 is referred as PG of 1 μM; PG2 is referred as PG of 2 μM; PG5 is referred as PG of 5 μM; PG10 is referred as PG of 10 μM.

TABLE 1
Treatment listing for the studies of the present invention

| Treatment No. | Pre-treated | Treated | Labeled |
|---|---|---|---|
| 1 | None | FU1 | FU1 |
| 2 | None | FU2 | FU2 |
| 3 | None | FU10 | FU10 |
| 4 | None | FU50 | FU50 |
| 5 | None | FU100 | FU100 |
| 6 | None | HCD0.5 | HCD0.5 |
| 7 | None | HCD1 | HCD1 |
| 8 | None | HCD2 | HCD2 |
| 9 | None | HCD5 | HCD5 |
| 10 | None | HCD10 | HCD10 |
| 11 | None | PG0.5 | PG0.5 |
| 12 | None | PG1 | PG1 |
| 13 | None | PG2 | PG2 |
| 14 | None | PG5 | PG5 |
| 15 | None | PG10 | PG10 |
| 16 | HCD0.5 | FU2 | HxF2 |
| 17 | HCD1 | FU2 | HyF2 |
| 18 | HCD2 | FU2 | HzF2 |
| 19 | PG0.5 | FU2 | PxF2 |
| 20 | PG1 | FU2 | PyF2 |
| 21 | PG2 | FU2 | PzF2 |

### Example 2

### Tests on Cell Viability

In this example, the effects of HCD, PG, and 5-FU on the viability of Caco2 cells and HT-29 cells were examined. The MTT assay was employed for this purpose. The MTT (3-(4-,5-dimethylthiazol-2-yl)-2,5-diphenyltetrazolium bromide) assay is a common colorimetric method in the field for cell viability analysis. MTT (yellow tetrazolium salt) is reduced to a purple formazan by living cells and detection to the purple formazan can be calculated as the cell viability.

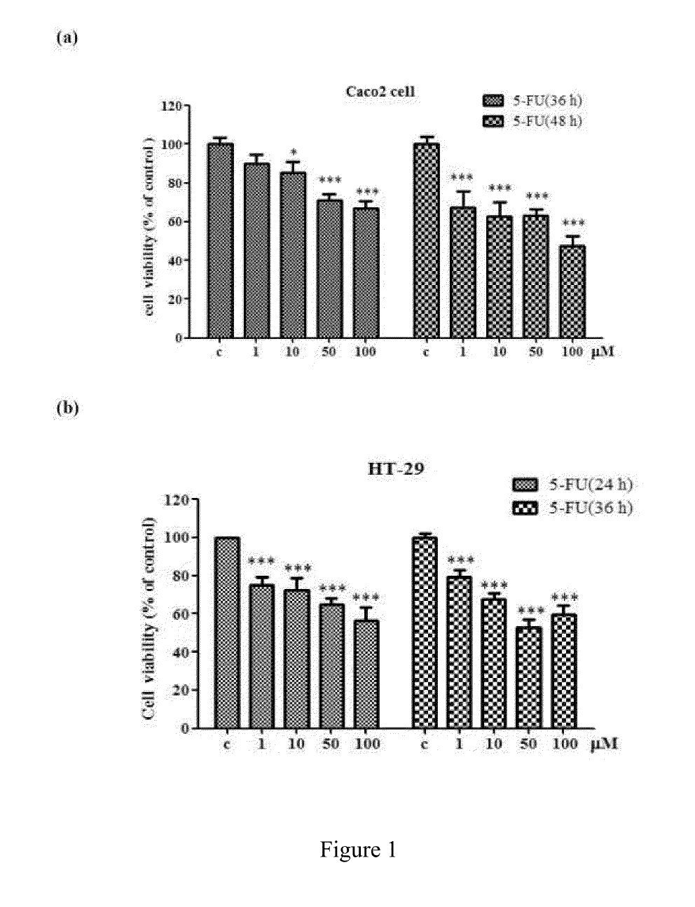

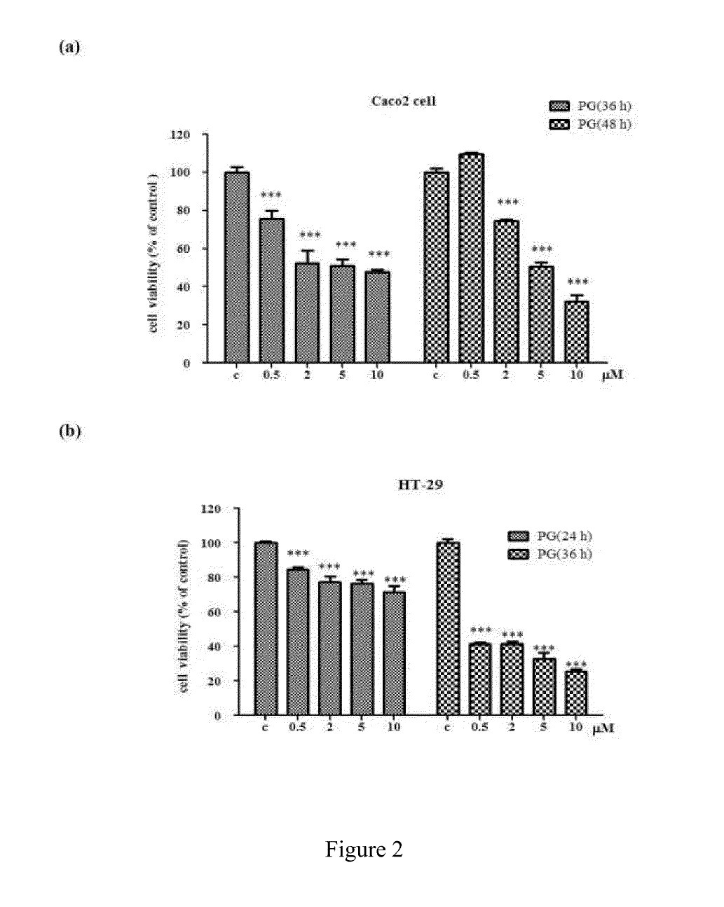

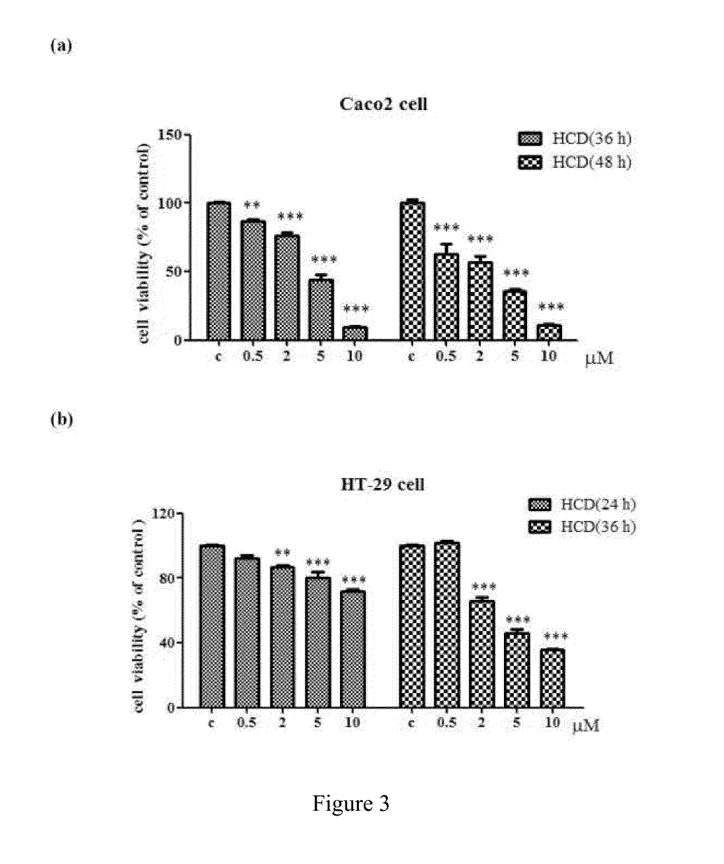

Briefly, cells (Caco2 or HT-29) were seeded in 96-well plate (7×10³ cell per well) and incubated overnight (37° C., 5% CO₂). Then, cells were treated for 24, 36, or 48 hr in accordance with the treatments listed in the Table 1. The results were showed in FIGS. 1, 2, 3.

The IC₅₀ of 5-FU was 100 μM and 50 μM in Caco2 cells (48 hr treatment) and HT-29 cells (36 hr treatment) respectively. The IC₅₀ of PG was 2 μM and 0.5 μM in Caco2 cells (48 hr treatment) and HT-29 cells (36 hr treatment) respectively. The IC₅₀ of HCD was 5 μM in Caco2 cells (48 hr treatment) and HT-29 cells (36 hr treatment) respectively. The results in FIGS. 1-3 indicated that the effective amount of PG or HCD was much less than that of 5-FU.

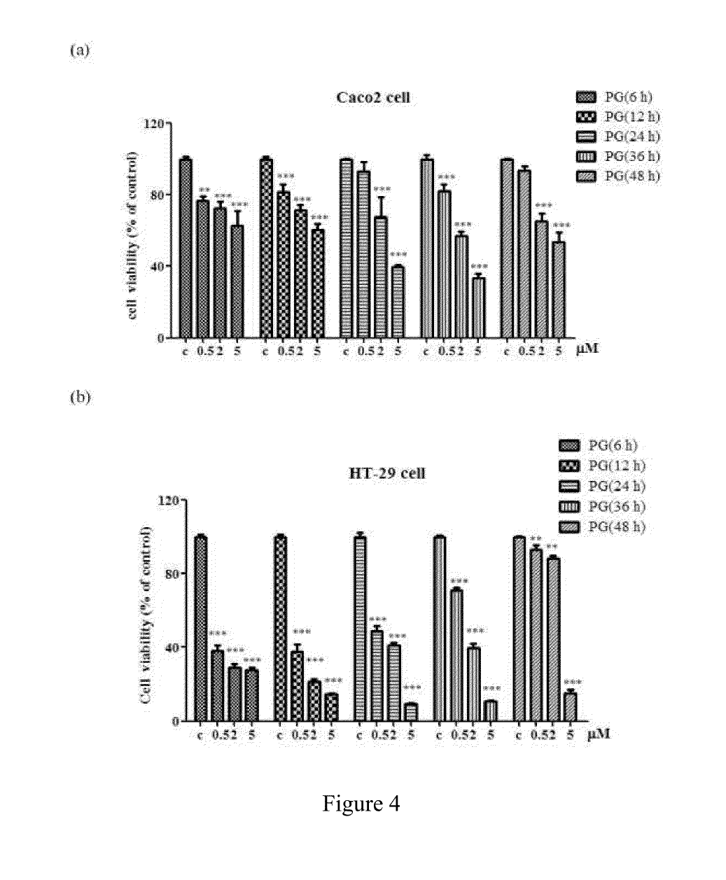

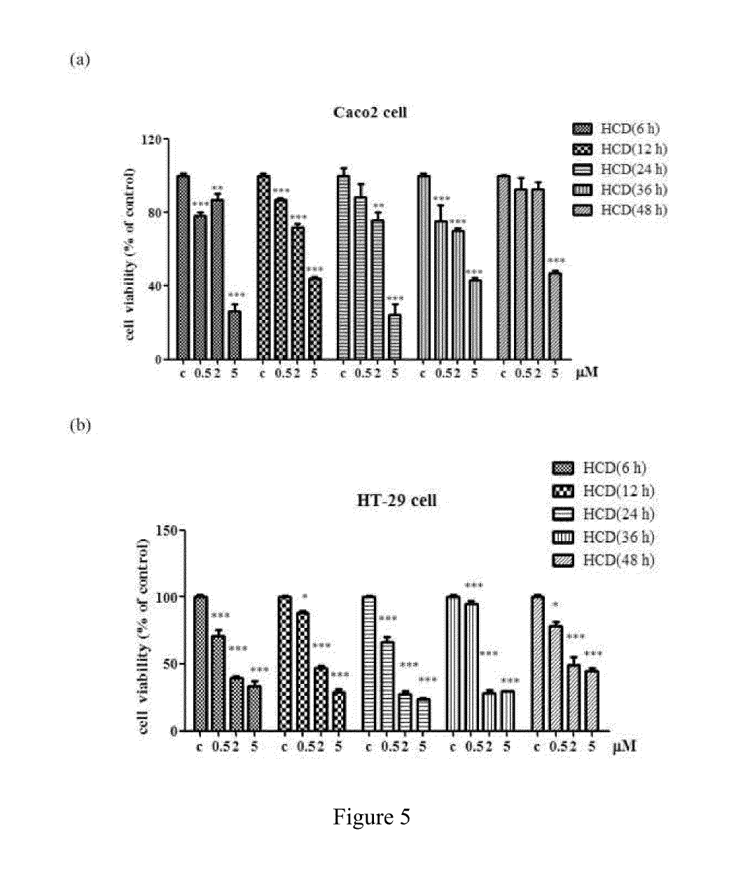

On the other hand, in the pretreatment analysis, cells (Caco2 or HT-29) were seeded in 96-well plate (7×10³ cell per well) and incubated overnight (37° C., 5% CO₂). In according to the treatments listed in the Table 1, Cells were pre-treated by HCD or PG for 6, 12, 24, 36, or 48 hr and then were treated by 5-FU for additional 24 hr. The results were showed in FIGS. 4 and 5.

The results in FIGS. 4 and 5 indicated that the strategy of pre-treating with PG or HCD significantly enhanced the efficacy of 5-FU in the subsequent treatment by lowering the effective amount to 2 μM.

In light of the foregoing, HCD showed reliable effects on the viability of colorectal cancer cells. Furthermore, our data indicated a pre-treatment strategy with a pre-treatment of HCD or PG before 5-FU treatment provided improved effects on colorectal cancer cells viability.

### Example 3

### Cell Cycle Analysis

The results of Example 2 in cell viability hinted the effects of HCD, PG, and 5-FU on arresting cell cycle. In this example, the phase of cell cycle of Caco2 and HT-29 cells after the treatment listed in Table 1 was determined by flow cytometer. Briefly, 7×10⁴ cells per well were inoculated in 12-wells plate and incubated overnight at 37° C., 5% CO₂. Then, cells were treated according to the treatment listed in Table 1 by indicated time period. After treatment, cells were harvested by trypsin and fixed with 70% ethanol at −20° C. for at least 3 hr. The cells were washed in cold PBS twice and then incubated with 1 mL (v/v) staining solution (20 μg/mL propidium iodide (PI), 0.1% Triton X-100, 0.2 mg/mL Rnase) at 37° C. for 30 minutes. Lastly, cells were analyzed by flow cytometer (Cytomics™ FC500, Backman, Fullerton, Calif., USA). Data from 10,000 cells were collected for each experimental group.

The following Tables 2 to 3 showed that the treatment of 5-FU increased cell cycle arrest at sub-G1 phase in both of the Caco2 and HT-29 cells. Tables 4 to 7 showed that, in both of the Caco2 and HT-29 cells, both of the treatment of HCD and PG increased cell cycle arrest at sub-G1 phase comparing with the control group in a dose- and time-dependent manner.

TABLE 2
Cell cycle distribution of Caco2 cells treated
with various dosages of 5-FU.
Caco2

| | | | | |
|---|---|---|---|---|
| Treatment | Sub G1 (%) | G0/G1 (%) | S (%) | G2/M (%) |

36 hr

| | | | | |
|---|---|---|---|---|
| Control | 5.97 ± 0.896 | 64.47 ± 6.01 | 13.4 ± 2.26 | 11.37 ± 2.0 |
| FU0.5 | 21.8 ± 5.09 | 46.15 ± 1.34** | 20.05 ± 1.91 | 8.65 ± 1.48** |
| FU2 | 40.1 ± 1.69*** | 36.65 ± 1.06*** | 17.1 ± 2.12 | 1.85 ± 0.5*** |
| FU5 | 42.6 ± 5.37*** | 39.1 ± 5.66*** | 12.5 ± 0.71 | 1.05 ± 0.21 |

48 hr

| | | | | |
|---|---|---|---|---|
| Control | 5.37 ± 1.91 | 65.2 ± 3.16 | 12.83 ± 1.69 | 11.83 ± 3.15 |
| FU0.5 | 20.9 ± 3.25 | 46.15 ± 1.34 | 13.65 ± 1.34 | 1.5 ± 0.14** |
| FU2 | 37.55 ± 14.64* | 35.35 ± 2.47*** | 18.05 ± 10.11 | 3.55 ± 3.89* |
| FU5 | 38.3 ± 24.47* | 36.7 ± 8.06*** | 17.5 ± 10.47 | 2.7 ± 2.95** |

Note:
1. Control group: untreated cells.
2. All the experiments were done in three independent trails and the data present as mean ± SD in triplicate determinations.
Compared at *p < 0.05, **p < 0.01, ***p < 0.01 levels with untreated control.

TABLE 3
Cell cycle distribution of HT-29 cells treated
with various dosages of 5-FU.
HT-29

| | | | | |
|---|---|---|---|---|
| Treatment | Sub G1 (%) | G0/G1 (%) | S (%) | G2/M (%) |

24 hr

| | | | | |
|---|---|---|---|---|
| Control | 4.8 ± 0.283 | 59.55 ± 0.354 | 13.5 ± 1.41 | 18.2 ± 1.273 |
| FU0.5 | 6.75 ± 0.35 | 40.05 ± 0.35* | 28.65 ± 1.20*** | 17.45 ± 3.04 |
| FU2 | 7.75 ± 0.21 | 50.3 ± 0.85 | 26.15 ± 1.77** | 9.15 ± 0.78* |
| FU5 | 6.85 ± 0.64 | 56.65 ± 6.01 | 21.45 ± 2.33** | 9.4 ± 3.68* |

36 hr

| | | | | |
|---|---|---|---|---|
| Control | 5.97 ± 0.896 | 64.47 ± 6.01 | 13.4 ± 2.26 | 11.37 ± 2.0 |
| FU0.5 | 21.8 ± 5.09 | 46.15 ± 1.34** | 20.05 ± 1.91 | 8.65 ± 1.48** |
| FU2 | 40.1 ± 1.69*** | 36.65 ± 1.06*** | 17.1 ± 2.12 | 1.85 ± 0.5*** |
| FU5 | 42.6 ± 5.37*** | 39.1 ± 5.66*** | 12.5 ± 0.71 | 1.05 ± 0.21 |

Note:
1. Control group: untreated cells.
2. All the experiments were done in three independent trails and the data present as mean ± SD in triplicate determinations.
Compared at *p < 0.05, **p < 0.01, ***p < 0.01 levels with untreated control.

TABLE 4
Cell cycle distribution of Caco2 cells treated
with various dosages of PG.
Caco2

| | | | | |
|---|---|---|---|---|
| Treatment | Sub G1 (%) | G0/G1 (%) | S (%) | G2/M (%) |

36 hr

| | | | | |
|---|---|---|---|---|
| Control | 8.3 ± 1.38 | 56.37 ± 4.44 | 15.83 ± 1.42 | 18 ± 3.24 |
| PG0.5 | 18.67 ± 2.06* | 51.3 ± 3.8 | 12.97 ± 0.49 | 14.93 ± 4.27 |
| PG2 | 21.47 ± 3.41** | 44.53 ± 7.05 | 14.33 ± 1.76 | 17.33 ± 1.95 |
| PG5 | 30.07 ± 6.7*** | 37.9 ± 6.04** | 14.23 ± 3.12 | 15.43 ± 7.86 |

48 hr

| | | | | |
|---|---|---|---|---|
| Control | 7.37 ± 1.08 | 42.73 ± 5.54 | 14.6 ± 1.73 | 21.27 ± 1.57 |
| PG0.5 | 11.67 ± 4.07 | 53.43 ± 13.12 | 18.17 ± 7.05 | 15.9 ± 8.23 |
| PG2 | 18.77 ± 7.49 | 49.87 ± 9.56 | 15 ± 6.14 | 12.43 ± 4.48 |
| PG5 | 33.53 ± 6.73*** | 45.83 ± 3.3 | 11.67 ± 2 | 8.7 ± 2.07* |

Note:
1. Control group: untreated cells.
2. All the experiments were done in three independent trails and the data present as mean ± SD in triplicate determinations.
Compared at *p < 0.05, **p < 0.01, ***p < 0.01 levels with untreated control.

TABLE 5
Cell cycle distribution of HT-29 cells treated
with various dosages of PG.
HT-29

| | | | | |
|---|---|---|---|---|
| Treatment | Sub G1 (%) | G0/G1 (%) | S (%) | G2/M (%) |

24 hr

| | | | | |
|---|---|---|---|---|
| Control | 4.8 ± 0.283 | 59.55 ± 0.354 | 13.5 ± 1.41 | 18.2 ± 1.273 |
| PG0.5 | 14.65 ± 4.03* | 65.3 ± 5.66*** | 5.9 ± 1.13** | 11.65 ± 0.78 |
| PG2 | 20.05 ± 5.87** | 54.35 ± 5.02 | 9.85 ± 1.2 | 12.35 ± 2.33 |
| PG5 | 19.6 ± 7.95** | 49.8 ± 7.07 | 12.9 ± 0 | 13.25 ± 0.21 |

36 hr

| | | | | |
|---|---|---|---|---|
| Control | 5.97 ± 0.896 | 64.47 ± 6.01 | 13.4 ± 2.26 | 11.37 ± 2.0 |
| PG0.5 | 37.88 ± 7.42*** | 48.28 ± 6.29* | 5.8 ± 0.68*** | 5.8 ± 0.84 |
| PG2 | 40.25 ± 3.32*** | 29.15 ± 3.18*** | 12.3 ± 1.13*** | 14.4 ± 0.71** |
| PG5 | 61.85 ± 2.47*** | 27.85 ± 1.06*** | 5.9 ± 0.42*** | 3.2 ± 0.85* |

Note:
1. Control group: untreated cells.
2. All the experiments were done in three independent trails and the data present as mean ± SD in triplicate determinations.
Compared at *p < 0.05, **p < 0.01, ***p < 0.01 levels with untreated control.

TABLE 6
Cell cycle distribution of Caco2 cells treated
with various dosages of HCD.
Caco2

| | | | | |
|---|---|---|---|---|
| Treatment | Sub G1 (%) | G0/G1 (%) | S (%) | G2/M (%) |

36 hr

| | | | | |
|---|---|---|---|---|
| Control | 8.3 ± 1.38 | 56.37 ± 4.44 | 15.83 ± 1.42 | 18 ± 3.24 |
| HCD0.5 | 14.4 ± 1.73 | 43.8 ± 3.81 | 17.3 ± 0.44 | 20.37 ± 4.24 |
| HCD 2 | 28.8 ± 4.2*** | 29.73 ± 4.22*** | 13.03 ± 0.5 | 23.73 ± 3.91 |
| HCD 5 | 51.77 ± 1.65*** | 19.4 ± 1.82*** | 9.9 ± 0.98** | 14.77 ± 0.8 |

48 hr

| | | | | |
|---|---|---|---|---|
| Control | 7.37 ± 1.08 | 42.73 ± 5.54 | 14.6 ± 1.73 | 21.27 ± 1.57 |
| HCD0.5 | 14.47 ± 5.05 | 44.13 ± 3.26 | 15.93 ± 0.9 | 21.2 ± 2.46 |
| HCD 2 | 36.17 ± 7.48*** | 25.73 ± 4.63 | 11.63 ± 1.08 | 22.03 ± 2.11 |
| HCD 5 | 54.2 ± 0.62*** | 15.67 ± 6.25** | 9.87 ± 0.55 | 12.9 ± 0.87 |

Note:
1. Control group: untreated cells.
2. All the experiments were done in three independent trails and the data present as mean ± SD in triplicate determinations.
Compared at *p < 0.05, **p < 0.01, ***p < 0.01 levels with untreated control.

TABLE 7
Cell cycle distribution of HT-29 cells treated
with various dosages of HCD.
HT-29

| | | | | |
|---|---|---|---|---|
| Treatment | Sub G1 (%) | G0/G1 (%) | S (%) | G2/M (%) |

24 hr

| | | | | |
|---|---|---|---|---|
| Control | 4.8 ± 0.283 | 59.55 ± 0.354 | 13.5 ± 1.41 | 18.2 ± 1.273 |
| HCD0.5 | 4.35 ± 0.495 | 82.9 ± 0.849** | 3.3 ± 0.424*** | 8.2 ± 0* |
| HCD 2 | 20.7 ± 0.707*** | 33.9 ± 1.414** | 17.95 ± 0.212 | 22 ± 1.414 |
| HCD 5 | 22.95 ± 7.99*** | 27.5 ± 4.808 | 15.1 ± 2.546 | 23.5 ± 2.97 |

36 hr

| | | | | |
|---|---|---|---|---|
| Control | 5.97 ± 0.896 | 64.47 ± 6.01 | 13.4 ± 2.26 | 11.37 ± 2.0 |
| HCD0.5 | 7.7 ± 0.81 | 59.25 ± 0.54 | 15.85 ± 1.68 | 14.25 ± 1.01* |
| HCD 2 | 43.95 ± 3.89*** | 35.95 ± 1.34*** | 7.8 ± 0.14** | 9.05 ± 1.485** |
| HCD 5 | 62 ± 6.93*** | 16.4 ± 4.95*** | 7.5 ± 1.13** | 11.15 ± 1.20*** |

Note:
1. Control group: untreated cells.
2. All the experiments were done in three independent trails and the data present as mean ± SD in triplicate determinations.
Compared at *p < 0.05, **p < 0.01, ***p < 0.01 levels with untreated control.

Please also refer to the following Tables 8-11. In terms of the pre-treatment strategy, it was noted that G2/M phase in Caco2 cells was decreased with 12 and 24 hr pretreatment of 1 or 2 μM of HCD and PG. While in HT-29 cells, pre-treatment of HCD or PG showed increase of sub G1 phase and decrease of G2/M phase. The results were consistent with the data obtained in Example 2, showing that the pre-treatment strategy is applicable for enhancing the effects of known drug, 5-FU.

TABLE 8
Cell cycle distribution of Caco2 cells pre-treated with various
dosages of PG and then treated with 5-FU (2 μM).
Caco2

| | | | | |
|---|---|---|---|---|
| Treatment | Sub G1 (%) | G0/G1 (%) | S (%) | G2/M (%) |

6 hr + 24 hr

| | | | | |
|---|---|---|---|---|
| Control | 4.75 ± 0.64 | 53.05 ± 2.47 | 15.75 ± 0.49 | 20.7 ± 1.13 |
| PxF2 | 10.65 ± 0.78 | 56.85 ± 0.49 | 12.75 ± 0.92 | 17 ± 0.57 |
| PyF2 | 13.65 ± 0.64 | 52.75 ± 4.31 | 14.35 ± 3.61 | 16.15 ± 0.07 |
| PzF2 | 16.75 ± 2.33 | 48.05 ± 1.91 | 14.65 ± 3.04 | 17.25 ± 2.76 |

12 hr + 24 hr

| | | | | |
|---|---|---|---|---|
| Control | 5.03 ± 1.56 | 51.93 ± 4.54 | 15.53 ± 1.07 | 22.3 ± 1.13 |
| PxF2 | 31.97 ± 17.45** | 45.27 ± 12.89 | 12.67 ± 2.75 | 7.1 ± 1.42*** |
| PyF2 | 50.7 ± 5.09** | 32.65 ± 4.88* | 7.45 ± 2.05* | 5.9 ± 0.14*** |
| PzF2 | 42.35 ± 1.34** | 36.55 ± 0.21 | 7.8 ± 1.84* | 7.8 ± 1.84*** |

24 hr + 24 hr

| | | | | |
|---|---|---|---|---|
| Control | 6.78 ± 1.29 | 49.5 ± 6.41 | 16.38 ± 2.55 | 21.12 ± 1.59 |
| PxF2 | 21.66 ± 6.67 | 50.23 ± 8.6 | 15.08 ± 0.51 | 9.23 ± 5.26*** |
| PyF2 | 41 ± 8.94** | 36.7 ± 5.9 | 11.37 ± 1.21* | 8.77 ± 2.5*** |
| PzF2 | 40.8 ± 8.34** | 34.9 ± 5.91 | 5.7 ± 0.71** | 11.65 ± 2.33** |

Note:
1. Control group: untreated cells.
2. All the experiments were done in three independent trails and the data present as mean ± SD in triplicate determinations.
Compared at *p < 0.05, **p < 0.01, ***p < 0.01 levels with untreated control.
3. 6 hr + 24 hr: 6 hr of pre-treatment and 24 hr of subsequent treatment.
4. 12 hr + 24 hr: 12 hr of pre-treatment and 24 hr of subsequent treatment.
5. 24 hr + 24 hr: 24 hr of pre-treatment and 24 hr of subsequent treatment.

TABLE 9
Cell cycle distribution of HT-29 cells pre-treated with various
dosages of PG and then treated with 5-FU (2 μM).
HT-29

| | | | | |
|---|---|---|---|---|
| Treatment | Sub G1 (%) | G0/G1 (%) | S (%) | G2/M (%) |

6 hr + 24 hr

| | | | | |
|---|---|---|---|---|
| Control | 2.7 ± 0.71 | 60.55 ± 0.07 | 13.9 ± 0.85 | 19.85 ± 0.92 |
| PxF2 | 37.9 ± 5.86 | 43.8 ± 4.75 | 9.4 ± 1.6 | 7.43 ± 1.69 |
| PyF2 | 32.05 ± 13.08 | 48.35 ± 11.10 | 9.25 ± 0.78 | 9.05 ± 1.06 |
| PzF2 | 29.6 ± 6.51 | 50.2 ± 6.36 | 11.15 ± 0.49 | 7.4 ± 0.85 |

12 hr + 24 hr

| | | | | |
|---|---|---|---|---|
| Control | 3.48 ± 0.94 | 53.53 ± 7.18 | 16 ± 3.99 | 22.13 ± 3.76 |
| PxF2 | 43.3 ± 1.56*** | 45.55 ± 0.07 | 3.4 ± 0.28** | 5.57 ± 0.07*** |
| PyF2 | 55.4 ± 2.55*** | 34.95 ± 2.05* | 2.9 ± 0.28** | 5.25 ± 0.64*** |
| PzF2 | 70.65 ± 7.42*** | 21.15 ± 8.41*** | 3.05 ± 1.2** | 3.8 ± 0.99*** |

24 hr + 24 hr

| | | | | |
|---|---|---|---|---|
| Control | 3.9 ± 1.09 | 60.45 ± 7.54 | 13.4 ± 4.47 | 16.13 ± 3.01 |
| PxF2 | 40.48 ± 6.36*** | 46.83 ± 10.12* | 5.58 ± 4.29* | 3.83 ± 0.31*** |
| PyF2 | 49.95 ± 4.34*** | 43.13 ± 3.69* | 2.43 ± 0.28** | 3.1 ± 0.37*** |
| PzF2 | 51.28 ± 4.72*** | 39.68 ± 4.01** | 3.5 ± 0.88** | 4.27 ± 0.55*** |

Note:
1. Control group: untreated cells.
2. All the experiments were done in three independent trails and the data present as mean ± SD in triplicate determinations.
Compared at *p < 0.05, **p < 0.01, ***p < 0.01 levels with untreated control.
3. 6 hr + 24 hr: 6 hr of pre-treatment and 24 hr of subsequent treatment.
4. 12 hr + 24 hr: 12 hr of pre-treatment and 24 hr of subsequent treatment.
5. 24 hr + 24 hr: 24 hr of pre-treatment and 24 hr of subsequent treatment.

TABLE 10
Cell cycle distribution of Caco2 cells pre-treated with various
dosages of HCD and then treated with 5-FU (2 μM).
Caco2

| | | | | |
|---|---|---|---|---|
| Treatment | Sub G1 (%) | G0/G1 (%) | S (%) | G2/M (%) |

6 hr + 24 hr

| | | | | |
|---|---|---|---|---|
| Control | 4.75 ± 0.64 | 53.05 ± 2.47 | 15.75 ± 0.49 | 20.7 ± 1.13 |
| HxF2 | 12.35 ± 2.33 | 55.4 ± 0.85 | 12.95 ± 1.63 | 16.1 ± 1.28 |
| HyF2 | 15.95 ± 3.32 | 51.7 ± 5.94 | 16.05 ± 5.44 | 13 ± 3.25 |
| HzF2 | 20.25 ± 6.72* | 45.7 ± 4.53 | 14.65 ± 4.03 | 16.4 ± 2.4 |

12 hr + 24 hr

| | | | | |
|---|---|---|---|---|
| Control | 5.03 ± 1.56 | 51.93 ± 4.54 | 15.53 ± 1.07 | 22.3 ± 1.13 |
| HxF2 | 21.13 ± 6.78 | 50.23 ± 4.63 | 15.2 ± 0.78 | 8.73 ± 3.07*** |
| HyF2 | 20.17 ± 2.66 | 51.6 ± 6.97 | 15.13 ± 4.47 | 8.67 ± 2.75*** |
| HzF2 | 28.13 ± 6.40* | 48.37 ± 8.16 | 11.6 ± 3.42* | 8.53 ± 7.7*** |

24 hr + 24 hr

| | | | | |
|---|---|---|---|---|
| Control | 6.78 ± 1.29 | 49.5 ± 6.41 | 16.38 ± 2.55 | 21.12 ± 1.59 |
| HxF2 | 19.48 ± 10.46 | 53.3 ± 8.42 | 13.68 ± 2.54 | 6.55 ± 7.08*** |
| HyF2 | 22.24 ± 9.69 | 48.14 ± 4.72 | 15.06 ± 4.66 | 10.72 ± 4.49*** |
| HzF2 | 35.67 ± 11.91* | 36.2 ± 5.76** | 10.78 ± 4.18** | 11.06 ± 3.74*** |

Note:
1. Control group: untreated cells.
2. All the experiments were done in three independent trails and the data present as mean ± SD in triplicate determinations.
Compared at *p < 0.05, **p < 0.01, ***p < 0.01 levels with untreated control.
3. 6 hr + 24 hr: 6 hr of pre-treatment and 24 hr of subsequent treatment.
4. 12 hr + 24 hr: 12 hr of pre-treatment and 24 hr of subsequent treatment.
5. 24 hr + 24 hr: 24 hr of pre-treatment and 24 hr of subsequent treatment.

TABLE 11
Cell cycle distribution of HT-29 cells pre-treated with various
dosages of HCD and then treated with 5-FU (2 μM).
HT-29

| | | | | |
|---|---|---|---|---|
| Treatment | Sub G1 (%) | G0/G1 (%) | S (%) | G2/M (%) |

6 hr + 24 hr

| | | | | |
|---|---|---|---|---|
| Control | 2.7 ± 0.71 | 60.55 ± 0.07 | 13.9 ± 0.85 | 19.85 ± 0.92 |
| HxF2 | 22.2 ± 0.43 | 53.5 ± 1.73 | 12.87 ± 0.47 | 8.83 ± 1.64 |
| HyF2 | 35.58 ± 15.56 | 40.1 ± 12.02 | 14.8 ± 4.24 | 9.85 ± 1.2 |
| HzF2 | 30.4 ± 3.69 | 48.47 ± 5.97 | 10.83 ± 0.67 | 8.37 ± 1.89 |

12 hr + 24 hr

| | | | | |
|---|---|---|---|---|
| Control | 3.48 ± 0.94 | 53.53 ± 7.18 | 16 ± 3.99 | 22.13 ± 3.76 |
| HxF2 | 29.6 ± 7.29*** | 46.67 ± 17.07 | 13.97 ± 6.40 | 9.07 ± 0.38*** |
| HyF2 | 36.4 ± 7.64*** | 41.13 ± 7.67 | 12.8 ± 0.75 | 11.4 ± 1.27*** |
| HzF2 | 51.4 ± 9.19*** | 34.35 ± 9.69* | 12.45 ± 0.07 | 3.1 ± 0.28*** |

24 hr + 24 hr

| | | | | |
|---|---|---|---|---|
| Control | 3.9 ± 1.09 | 60.45 ± 7.54 | 13.4 ± 4.47 | 16.13 ± 3.01 |
| HxF2 | 11.47 ± 2.4 | 60.93 ± 9.02 | 16.4 ± 4.03 | 8.7 ± 0.57** |
| HyF2 | 16.23 ± 1.7** | 71.6 ± 6.55** | 4.3 ± 2.31* | 6.83 ± 3.39** |
| HzF2 | 33.3 ± 6.37*** | 44.7 ± 2.54 | 7.55 ± 0.63 | 12.5 ± 2.83* |

Note:
1. Control group: untreated cells.
2. All the experiments were done in three independent trails and the data present as mean ± SD in triplicate determinations.
Compared at *p < 0.05, **p < 0.01, ***p < 0.01 levels with untreated control.
3. 6 hr + 24 hr: 6 hr of pre-treatment and 24 hr of subsequent treatment.
4. 12 hr + 24 hr: 12 hr of pre-treatment and 24 hr of subsequent treatment.
5. 24 hr + 24 hr: 24 hr of pre-treatment and 24 hr of subsequent treatment.

### Example 4

### Mouse Model Establishment

A mouse model bearing inflammatory bowel disease (IBD) is a common and reliable animal model for in vivo cancer research. However, the conventional mouse model bearing IBD is not ideal because it requires several months to establish the mouse model. In addition, another concern of the conventional mouse model is that those mouse models are established in mice with immunodeficiency, which means the physiological condition of the experimental animals are not “normal” and therefore have more variance than expecting. In this example, a mouse model was established in a “normal mouse” by a more time-efficiency manner.

The animal experiments were approved by the National Dong-Hwa University Animal Ethics Committee and the experimental protocols were used according to the “Guide for the Care and Use of Laboratory Animals” of National Dong-Hwa University. On day 0, C57/BL6 mice (6-8 weeks old) were weighted and injected (intraperitoneal; i.p.) with 10 mg/kg body weight of azoxymethane (AOM) and then fed with 2% Dextran sodium sulfate solution (DSS solution) via water every day for 7 days. On day 8, the 2% DSS solution was changed to normal water for another 7 days. The cycle of DSS solution and water was repeated and the induction procedure was completed on day 35.

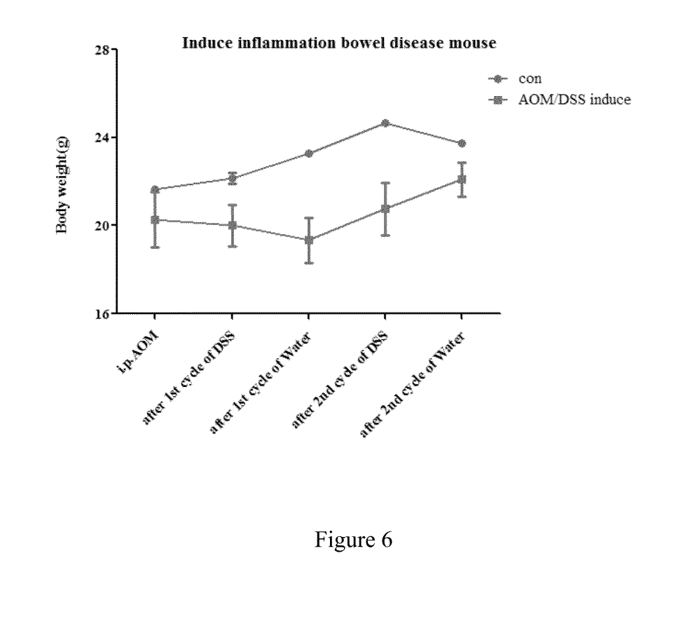

During the induction procedure, the body weight of the experimental mice was recorded and compared with mice of control group. The results were showed in FIG. 6. The body weight of experimental mice was lighter than that of the control mice. However, as the body weight of the experimental mice increased stably, it can be construed that the induction procedure did not significantly affect the physiological condition of the animals.

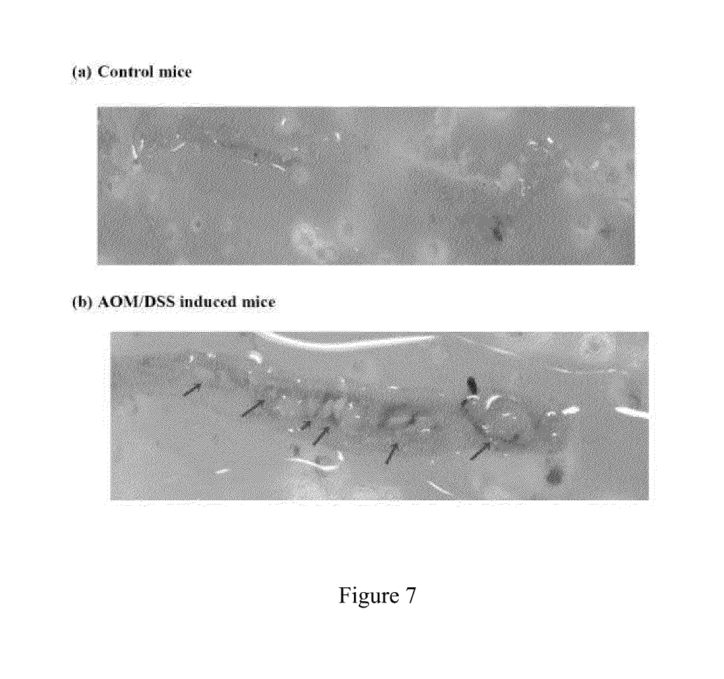

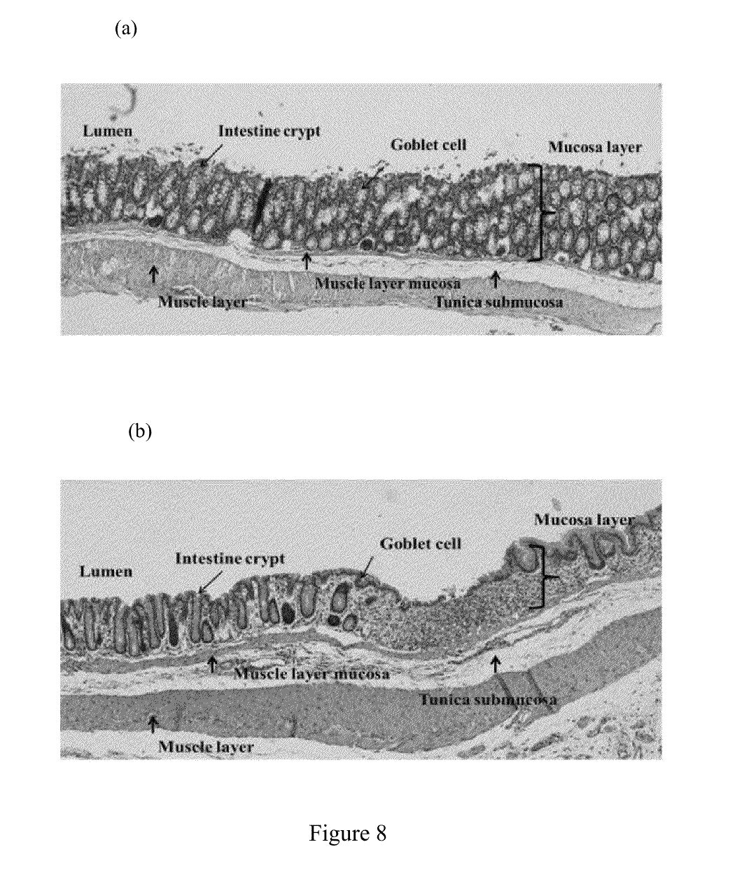

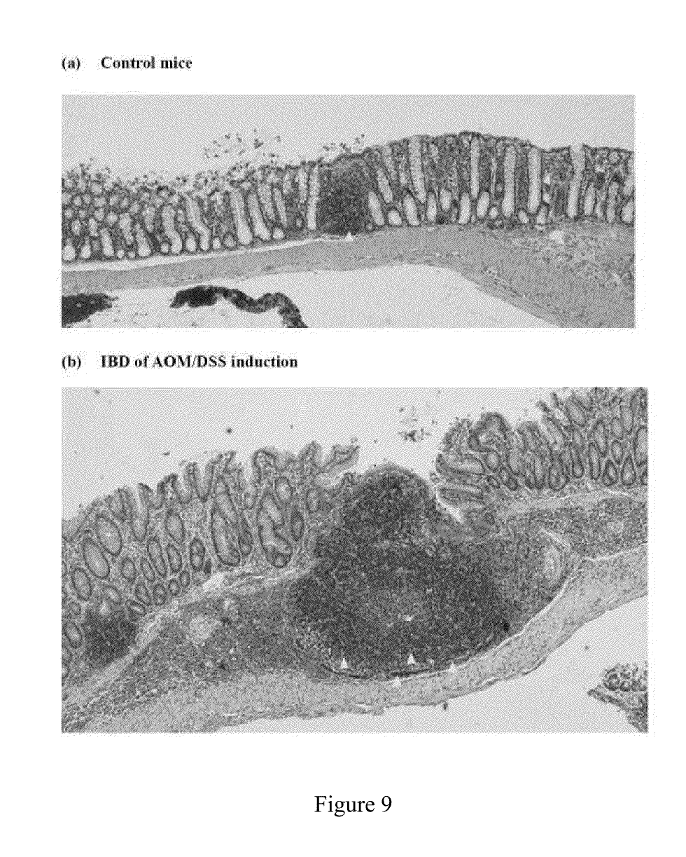

On day 35, the mice were sacrificed. The colon, spleen, liver, and kidney thereof were immediately removed, observed, and further examined by H&E staining. FIG. 7 showed the morphology of the colorectal portion of control mice and experimental mice. The colorectal portion of the experimental mice had some polypus and enlargement lymph nodes (arrows). Moreover, the H&E staining showed that, comparing with the control mice, the intestine of the experimental mice showed irregular arrangement and shorter villus and the mucous, muscle mucosa and muscle layer thereof were thicker than that of control mice (FIG. 8). Furthermore, the lymph nodes of the experimental mice were enlarged and infiltrated into the muscle mucosa layer in comparison with that of normal mice (FIG. 9).

The aforesaid data showed that the induction procedure was success in establishing IBD in the experimental mice.

### Example 5

### Examination to the Efficacy of HCD in Colorectal Cancer Treatment by Mouse Model

After the establishment of IBD in the experimental mice (on day 35), the mice were injected (i.p.) with 5-FU (15 mg/kg body weight) or HCD (0.64, 1.6, or 6.4 mg/kg body weight) once every three days for additional 30 days. Then, the mice were sacrificed. The colon, spleen, liver, and kidney thereof were immediately removed, observed, and further examined by H&E staining.

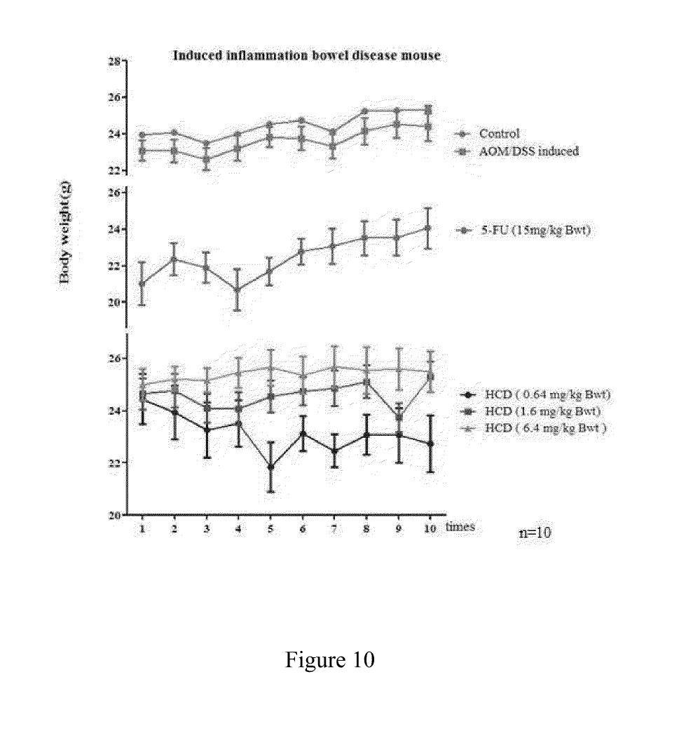

During the experiment period, the body weight of the mice was recorded. The result in FIG. 10 showed that the body weight of mice treated with 5-FU and low dose of HCD (0.64 mg/kg body weight) was initially reduced. Nevertheless, the body weight of all mice was progressively increased or maintained stably during the experiment.

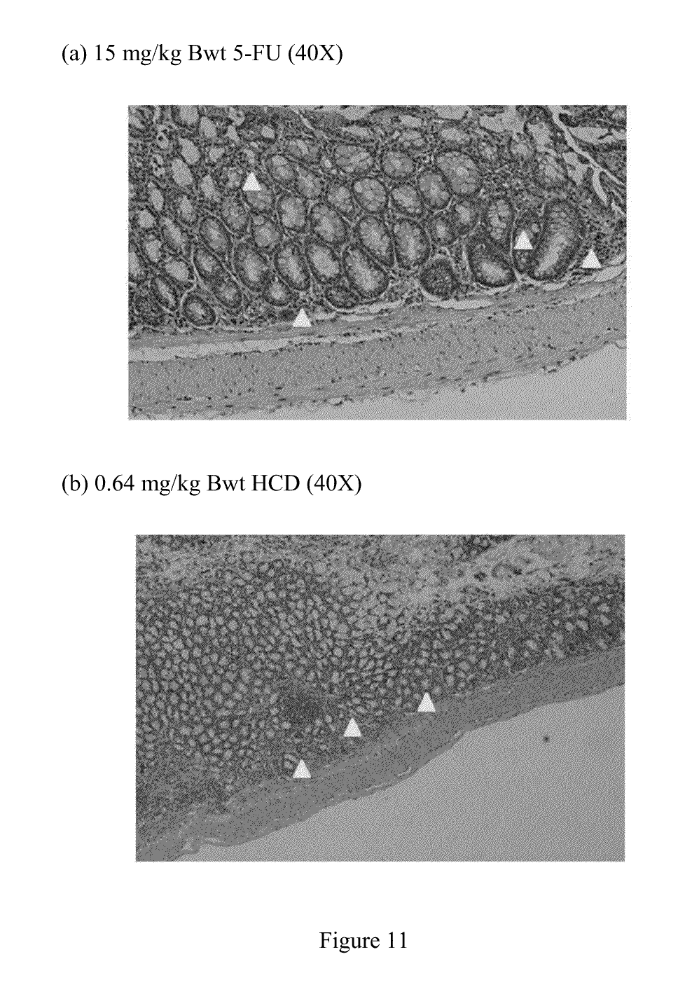

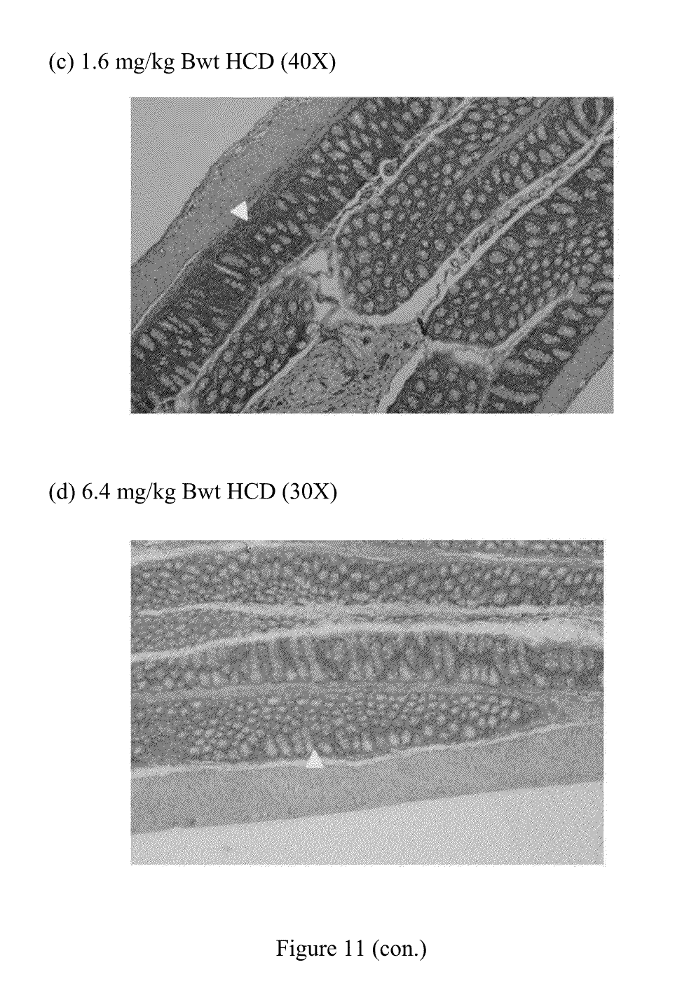

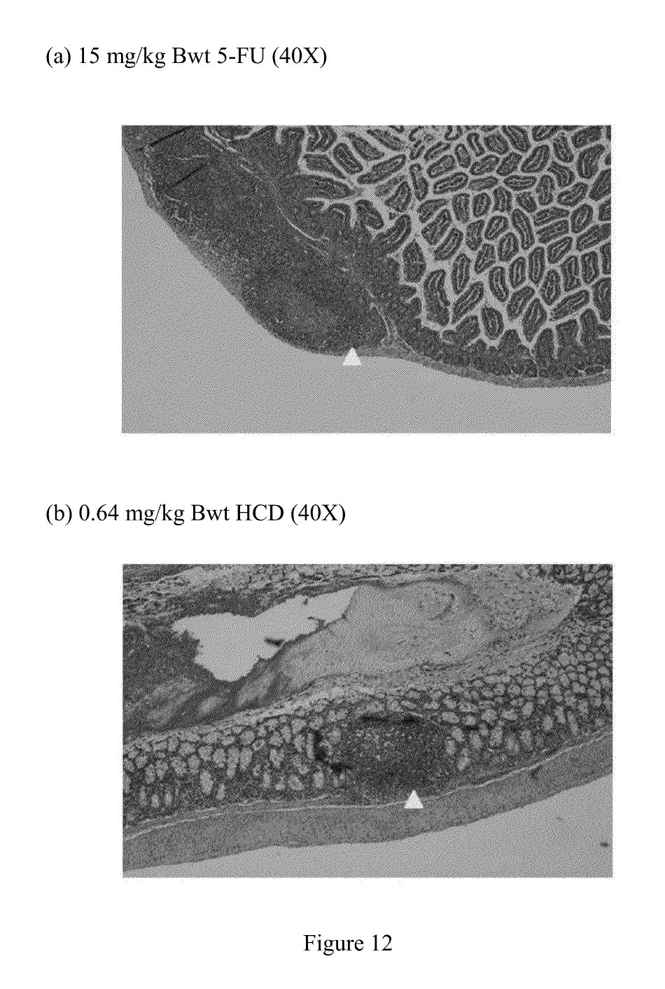

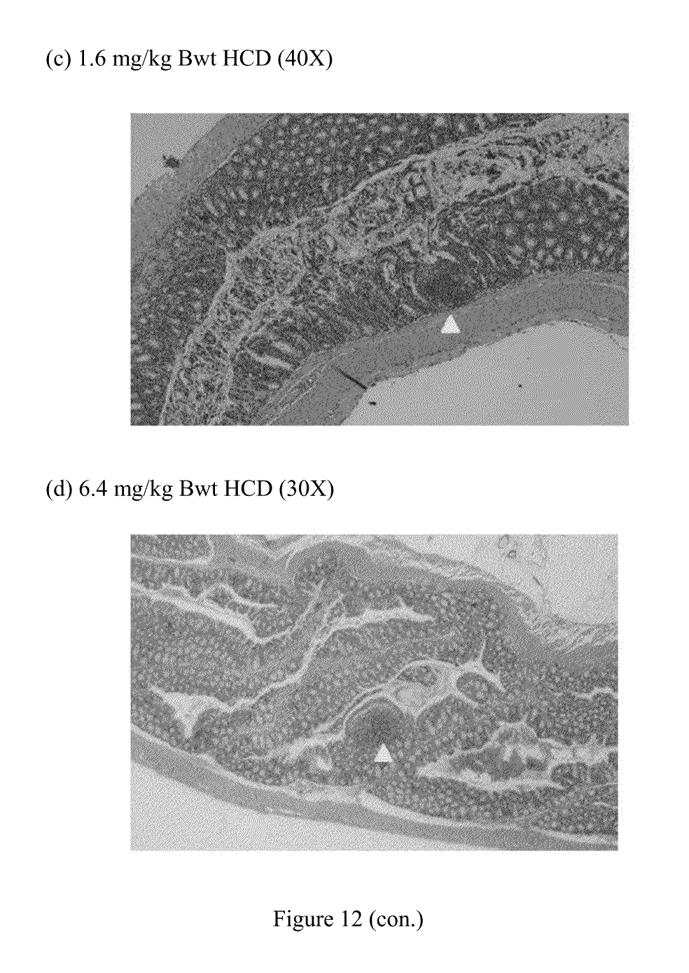

The H&E staining of the intestine vertical section of the mice were showed in FIGS. 11 and 12. The H&E staining results showed that the intestine irregular arrangement of villus were observed in mouse treated with 15 mg/kg body weight of 5-FU and 0.64 mg/kg body weight of HCD (FIG. 11). The lymph node was enlarged in mouse treated with 15 mg/kg body weight of 5-FU, but not infiltrated into muscle mucosa layer in mouse treated with 0.64 mg/kg body weight of HCD (FIG. 12). On the other hand, the intestine of villus of mouse treated with 1.6 or 6.4 mg/kg body weight of HCD were arranged in neat rows nearly as seen in the control mice (FIG. 11). Also, the lymph nodes of mouse treated with 1.6 or 6.4 mg/kg body weight of HCD were not infiltrated into muscle mucosa layer (FIG. 12). The above evidence confirmed the efficacy of HCD in treating colorectal cancer in vivo.

1. A pharmaceutical composition for colorectal cancer treatment, comprising:
an effective amount of 16-hydroxy-cleroda-3,13-dine-15,16-olide; and
a pharmaceutically acceptable carrier.

2. The composition of claim 1, wherein said effective amount is 0.6 to 6.5 mg/kg body weight.

3. The composition of claim 1, comprising 0.5 to 10 μM of said 16-hydroxy-cleroda-3,13-dine-15,16-olide.

4. The composition of claim 1, wherein said pharmaceutically acceptable carrier is water, phosphate buffered saline, alcohol, glycerol, chitosan, alginate, chondroitin, Vitamin E, mineral oil, dimethyl sulfoxide (DMSO), or a combination thereof.

5. The composition of claim 1, wherein an administration route of said composition is via oral administration, intravenous injection, intrathecal injection, intraperitoneal injection, or a combination thereof.

6. A method for treating a colorectal cancer, comprising:
administrating an object in need an effective amount of 16-hydroxy-cleroda-3,13-dine-15,16-olide.

7. The method of claim 6, wherein said effective amount is 0.6 to 6.5 mg/kg body weight.

8. The method of claim 6, wherein said administrating is via oral administration, intravenous injection, intrathecal injection, intraperitoneal injection, or a combination thereof.

9. The method of claim 6, wherein said 16-hydroxy-cleroda-3,13-dine-15,16-olide is administrated with a pharmaceutically acceptable carrier.

10. The method of claim 9, wherein said pharmaceutically acceptable carrier is water, phosphate buffered saline, alcohol, glycerol, chitosan, alginate, chondroitin, Vitamin E, mineral oil, dimethyl sulfoxide (DMSO), or a combination thereof.

11. The method of claim 6, further comprising a step after administrating said 16-hydroxy-cleroda-3,13-dine-15,16-olide: administrating said object with 5-Fluorouracil.

12-19. (canceled)
